(19) United States
(12) Patent Application Publication
(10) Pub. No.: US 2009/0085567 A1
(43) Pub. Date: Apr. 2, 2009

(54) MAGNETIC RESONANCE APPARATUS WITH SHIM ARRANGEMENT

(76) Inventors: Ralph Kimmlingen, Nuernberg (DE); David Melotte, Allen (GB); Wolfgang Renz, Erlangen (DE); Ian Wilkinson, Witney (GB)

Correspondence Address: SCHIFF HARDIN, LLP;PATENT DEPARTMENT, 6600 SEARS TOWER, CHICAGO, IL, 60606-6473, US

(21) Appl. No.: 12/243,044

(22) Filed: Oct. 1, 2008

(30) Foreign Application Priority Data
Oct. 1, 2007 (DE) 10 2007 047 023.3

(51) Int. Cl. G01R 33/32; A61B 5/055

(52) U.S. Cl. 324/318; 600/422

(57) ABSTRACT

A magnetic resonance apparatus has an examination region to accommodate a patient to be examined, and a body coil that circumferentially encompasses the examination region and is designed for magnetic resonance examination of the patient. A gradient coil circumferentially encompasses the examination region and the body coil and is designed to detect the position of magnetic resonance measurement values. A basic field magnet is designed to form a basic magnetic field in the examination region for a patient examination to be conducted. The basic field magnet at least partially encompasses the examination region, the body coil and the gradient coil. A shim device is used that is designed to influence the basic magnetic field. Components of the shim device and components of the body coil are associated to exhibit a common distance relative to the longitudinal axis of symmetry of the examination region and thus encompass the examination region.

### BACKGROUND OF THE INVENTION

1. Field of the Invention

This invention concerns a magnetic resonance apparatus having a having a shim arrangement for shimming the basic magnetic field.

2. Description of the Prior Art

It is presently sought to design magnetic resonance apparatuses in an optimally space-saving manner. Opposing this minimization are, in addition to the actual magnetic field components, devices that are used to compensate tolerances in the basic field magnets. In particular active and/or passive shim devices that occupy additional volume are required for this purpose.

Passive shim devices use metal elements known as shim irons that are arranged at least in part in the region of the gradient coil in conventional apparatuses.

Active shim devices are fashioned as electrically conductive coils that can likewise be arranged at least in part in the region of the gradient coil.

FIG. 3 shows the basic design of a magnetic resonance apparatus according to the prior art in cross-section.

The apparatus has an examination region BORE into which a patient to be examined can be inserted, as well as a region BGF demarcating the bottom of the examination region BORE.

The examination region BORE is encompassed by a body coil BC that is used for the actual examination. The body coil BC is fashioned, for example, as a transmission antenna structure in the form of a birdcage antenna. Its rod-shaped elements encompass the examination region BORE parallel to one another and are therefore arranged parallel to the cylinder wall of the examination region BORE.

A gradient coil GC is used to associate measurement values with positions. The gradient coil GC has three sub-coils for a three-dimensional position specification, respectively for the x-, y- and z-coordinates or axial segments thereof.

A basic field magnet GFM provides a strong basic magnetic field required for the nuclear spin alignment. In order to compensate for unwanted deviations of the basic magnetic field within the examination region BORE, shim devices SE are provided in the region of the gradient coil GC. The (bar-shaped) passive shim irons can be inserted into spaces or receptacles known as shim drawers at predetermined positions in the region of the gradient coil GC.

The respective modules are arranged in overlapping planes Ex in the cross-section representation.

A component of the basic field magnet GFM is arranged in a ground plane E0 while both components of the gradient coil GC and components of the shim device SE are arranged in a plane E1 above the ground plane E0.

Components of the body coil BC are arranged in an additional plane E2 while a plane E3 above this contains the bounding region BGF.

The shim irons used in the shim device SE are magnetized with a time-dependent variation. This variation is designated as a “shim drift” and is ultimately caused by pulses that are radiated via the gradient coil GC.

To reduce the “shim drift”, it is known to use superconducting shim devices, but these are very complicated and expensive.

As an alternative, cobalt-containing shim irons are used that are likewise expensive and exhibit only a limited effect against the “shim drift.”

It is also possible for the mass of the shim irons to be reduced, but this means that as a countermeasure the spatial dimensions of the magnetic resonance apparatus must increase.

As an alternative it is known to arrange the shim irons within the examination region BORE, but this means the diameter of the examination region BORE must then be enlarged.

### SUMMARY OF THE INVENTION

An object of the present invention is to provide a magnetic resonance apparatus in which a reduction of the space requirement is possible, wherein magnetic characteristics of previous apparatuses are at least retained.

This object is achieved in accordance with the invention by a magnetic resonance apparatus that has an examination region to accommodate a patient to be examined, a body coil that circumferentially encompasses the examination region and is designed for magnetic resonance examination of the patient, a gradient coil that circumferentially encompasses the examination region and the body coil and is designed to detect the position of magnetic resonance measurement values, a basic field magnet that generates a basic magnetic field in the examination region for a patient examination to be conducted, with the basic field magnet at least partially encompassing the examination region, the body coil and the gradient coil. A shim device is used in order to influence the basic magnetic field in the examination region.

Both components of the shim device and components of the body coil are associated with a common plane and two-dimensionally encompass the examination region.

In other words, the components of the shim device and the components of the body coil exhibit a common distance relative to the longitudinal axis of symmetry of the examination region.

In a typical cylindrical examination region, the components of the shim device and the components of the body coil are arranged on a cylindrical surface, the cylindrical surface encompassing the examination region.

In the arrangement according to the invention it was realized that an advantageous positioning of the shim device is provided by making the body coil used for examination significantly shorter than the gradient coil required for localization.

In the arrangement according to the invention, the shim device is displaced from the region of the gradient coil in the direction of the examination region, more precisely into the region of the body coil. This achieves a volume reduction of the gradient coil.

By virtue of the arrangement of the shim device according to the invention, the elements thereof (for example the shim irons) are exposed to a reduced “shim drift.” Cooling devices that were previously required for cooling the shim device are reduced in complexity or can even be omitted entirely.

A constant formation of the basic magnetic field in the examination region is enabled via the displacement of the shim device into the region of the body coil. The development of eddy currents in the shim device and heating of its components that results therefrom, and volume and position changes of the shim components that are caused by the heating, are minimized.

In an embodiment, the shim device is decoupled from the body coil or from its components with the use of a radio-frequency shield. A tuning of the body coil can therefore be implemented that is no longer influenced by the elements of the shim device in the further course of the procedure.

The radio-frequency shield is preferably metallically designed and thus forms an electrically conductive layer.

The shim device can be cooled, for example, with the use of water cooling.

### BRIEF DESCRIPTION OF THE DRAWINGS

FIG. 1 shows a first exemplary embodiment of the arrangement according to the invention.

FIG. 2 shows a second exemplary embodiment of the arrangement according to the invention.

FIG. 3 shows the basic design of a magnetic resonance apparatus according to the prior art as described above.

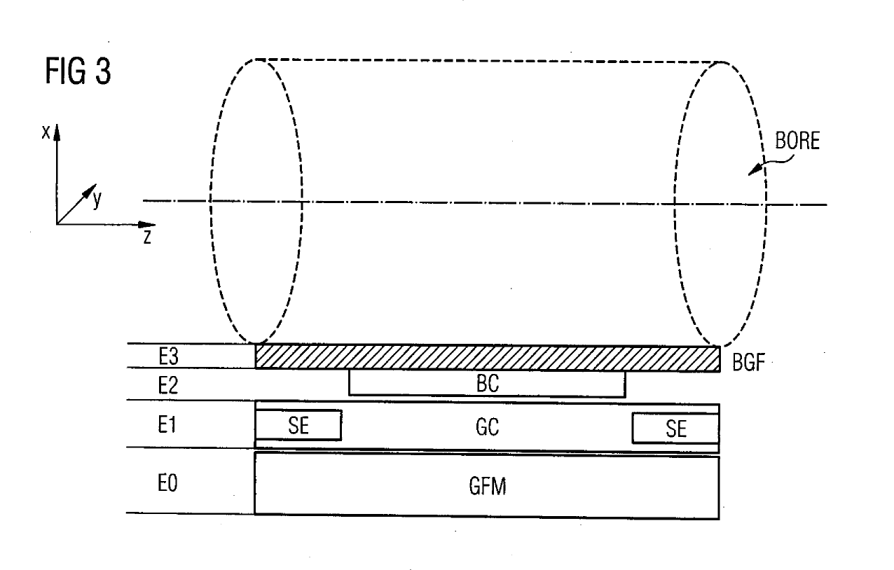

### DESCRIPTION OF THE PREFERRED EMBODIMENTS

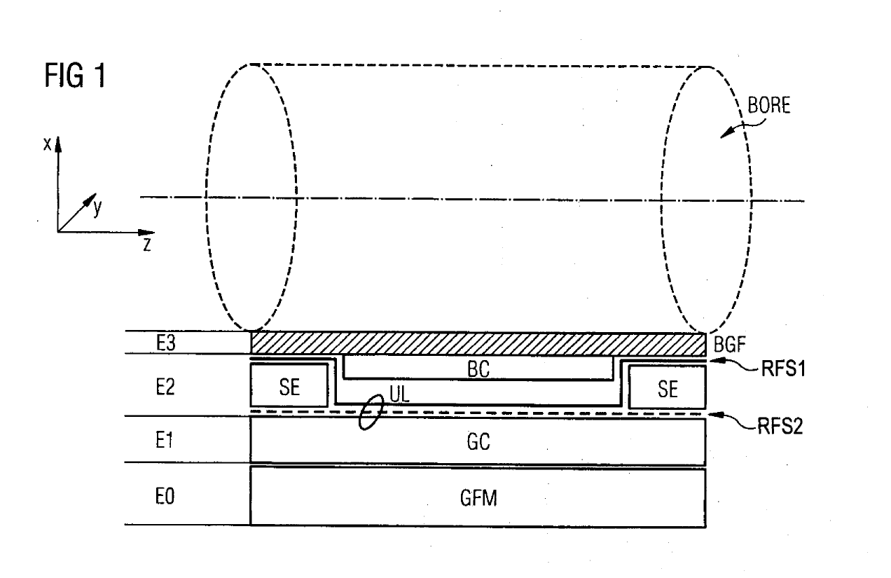

FIG. 1 shows a first exemplary embodiment of the arrangement according to the invention in a significantly simplified cross-section representation.

A cylindrical examination region BORE into which a patient to be examined can be introduced is terminated towards the bottom by a bounding region.

The examination region BORE is encompassed by what is known as a body coil BC that is used for the actual examination.

The body coil BC is fashioned as a transmission antenna structure in the form of a birdcage antenna, for example. The rod-shaped elements thereof are parallel to one another and encompass the examination region BORE and are therefore arranged parallel to the wall of the cylindrical examination region BORE.

A gradient coil GC is used to associate measurement values with positions. The gradient coil GC has three sub-coils (not shown in detail here) for a three-dimensional position specification, namely respective sub-coils to specify x-, y- and z-coordinates or axial segments thereof.

A basic magnetic field GFM provides a strong basic magnetic field required for the nuclear spin alignment.

According to the invention, respective shim devices SE are provided in the region of the body coil BC in order to compensate for unwanted deviations of the basic magnetic field within the examination region BORE.

These shim devices can use shim irons, for example, or be designed as electrical shim coils.

In a simplified cross-section image, the respective modules are arranged in planes Ex lying atop one another.

A component of the basic field magnet GFM is arranged in a ground plane E0 while components of the gradient coil GC are arranged in a plane E1.

In addition to the components of the body coil BC, the shim devices SE are likewise arranged in an additional plane E2 that lies above the plane E1.

A plane E3 above this contains the bounding region BGF.

The components of the shim device SE and the components of the body coil BC thus exhibit a common (same) distance relative to the longitudinal axis of symmetry of the cylindrical examination region BORE and encompass the examination region BORE.

The shown shim devices SE outwardly bound the body coil BC or, respectively, bound it in the direction of a z-axis that extends in the longitudinal direction of the cylindrical examination region BORE.

In a preferred development, a conductive radio-frequency shield RFS1 is arranged between the components of the shim device SE and the body coil BC. A tuning of the body coil BC is thereby possible that is independent of the elements of the shim device SE.

An additional radio-frequency shield RFS2 can also be used to decouple the gradient coil GC from the components of the shim device SE.

The two radio-frequency shields RFS1 and RFS2 are advantageously merged in an overlap region UL.

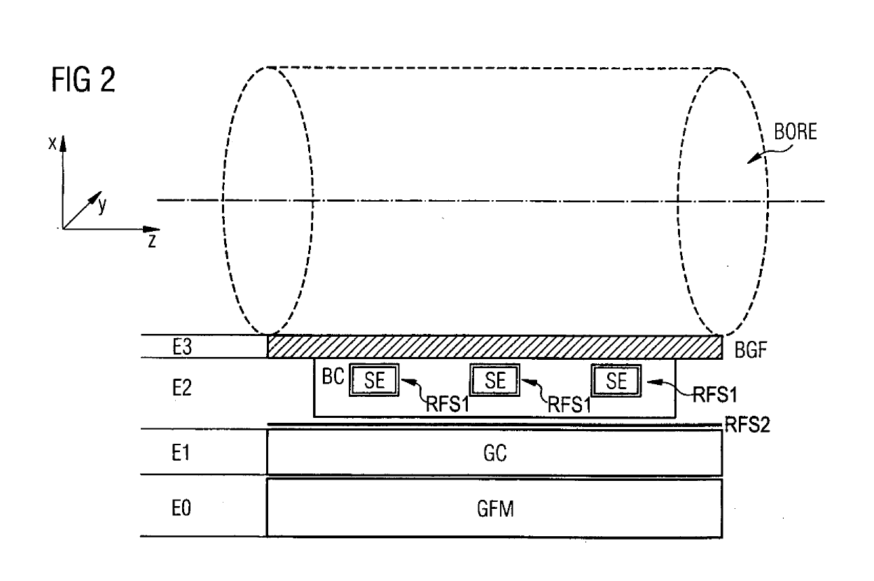

FIG. 2 shows a second exemplary embodiment of the arrangement according to the invention in a significantly simplified cross-section representation.

Relative to FIG. 1, the shim devices SE shown here do not form external bounds of the body coil BC or, respectively, bounds in the z-direction; rather, they are designed as an integrated part of the body coil BC.

In a preferred embodiment, a conductive radio-frequency shield RFS1 is respectively arranged between each component of the shim device SE and the body coil BC. Tuning of the body coil BC that is independent of the elements of the shim device SE is thereby possible.

An additional radio-frequency shield RFS2 decouples the gradient coil GC from the body coil BC.

The two examples presented in FIG. 1 and FIG. 2 represent only some of the possibilities for positioning the shim devices SE relative to the body coil BC; arbitrary mixed forms are possible.

Although modifications and changes may be suggested by those skilled in the art, it is the intention of the inventor to embody within the patent warranted hereon all changes and modifications as reasonably and properly come within the scope of his contribution to the art.

We claim as our invention:

1. A magnetic resonance apparatus comprising:
a magnetic resonance scanner having an examination region configured to accommodate a patient therein;
a radio-frequency body coil that circumferentially encompasses said examination region for interacting with a patient in the examination region to acquire magnetic resonance data therefrom;
a gradient coil that circumferentially encompasses the examination region and the body coil to detect a position of magnetic resonance signals emitted by the patient during said acquisition of magnetic resonance data;
a basic field magnet that generates a basic magnetic field in the examination region during said acquisition of magnetic resonance data, said basic field magnet at least partially encompassing said examination region and said body coil and said gradient coil;
a shim device in said scanner that influences the basic magnetic field to homogenize at least a portion of said basic magnetic field; and
said shim device being comprised of shim device components and said body coil being comprised of body coil components, and said shim device components and said body coil components being disposed at a same distance relative to a longitudinal axis of symmetry of the examination region so as to encompass the examination region.

2. A magnetic resonance apparatus as claimed in claim 1 comprising a conductive radio-frequency shield between said shim device components and said body coil components.

3. A magnetic resonance apparatus as claimed in claim 1 wherein said shim device is integrated into said body coil.

4. A magnetic resonance apparatus as claimed in claim 1 wherein said shim device externally terminates said body coil along said longitudinal axis of said examination region.

5. A magnetic resonance apparatus as claimed in claim 1 wherein said examination region has a cylindrical shape.

6. A magnetic resonance apparatus as claimed in claim 1 wherein said body coil is a birdcage antenna.

7. A magnetic resonance apparatus as claimed in claim 1 wherein said shim device components are shim irons.

8. A magnetic resonance apparatus as claimed in claim 1 wherein said shim device components are electrical shim coils.
